(19) United States
(12) Patent Application Publication
(10) Pub. No.: US 2006/0118650 A1
(43) Pub. Date: Jun. 8, 2006

(54) Scent release massager

(76) Inventor: Chong-Fa Yu, Panchiao City (TW)

Correspondence Address: LIN & ASSOCIATES INTELLECTUAL PROPERTY, P.O. BOX 2339, SARATOGA, CA, 95070-0339, US

(21) Appl. No.: 11/004,293

(22) Filed: Dec. 3, 2004

(51) Int. Cl. A24F 25/00; A61L 9/04

(52) U.S. Cl. 239/34; 239/47; 239/55

(57) ABSTRACT

A scent release massager includes a container with multiple slits defined in a side wall thereof, a casing received in the container and supported by a fan, a motor securely received in the casing to be supported by the fan, a baffle sandwiched between the fan and the bottom periphery defining the bottom opening to support thereon scent particles and a cover detachably connected to the container to close the top opening of the container. When the motor is activated, the blades of the fan is driven to rotate so that scent from the scent particles is able to escape from the slits of the container.

### BACKGROUND OF THE INVENTION

1. Field of the Invention

The present invention relates to a scent release massager, and more particularly to the scent release massager to allow the user to have muscle and spiritual relief simultaneously.

2. Description of Related Art

In general, a massager is designed to release tensioned muscle and nothing else. That is, the massager, no matter what specific design it has on the physical body, has only one function.

Soothing scent is able to allow people surrounding the scent to have mental and spiritual relax. However, the soothing scent is not able to relieve physical sour or fatigue.

Other objects, advantages and novel features of the invention will become more apparent from the following detailed description when taken in conjunction with the accompanying drawings.

### SUMMARY OF THE INVENTION

The primary objective of the present invention is to provide an improved a scent release massager that can relieve user's muscle fatigue and allow the user to have mental and spiritual relax simultaneously.

Another objective of the present invention is to use a decorative enclosure to receive therein the scent release massager so that when the scent release massager is not in use, the decorative enclosure is able to function as a decorator.

Other objects, advantages and novel features of the invention will become more apparent from the following detailed description when taken in conjunction with the accompanying drawings.

### BRIEF DESCRIPTION OF THE DRAWINGS

FIG. 1 is an exploded perspective view of the present invention;

FIG. 2 is a perspective view of the scent release massager in FIG. 1;

FIG. 3 is a perspective view of a second embodiment of the scent release massager of the present invention;

FIG. 4 is a perspective view of a third embodiment of the scent release massager of the present invention; and

FIG. 5 is a perspective view of a fourth embodiment of the scent release massager of the present invention.

### DETAILED DESCRIPTION OF PREFERRED EMBODIMENT

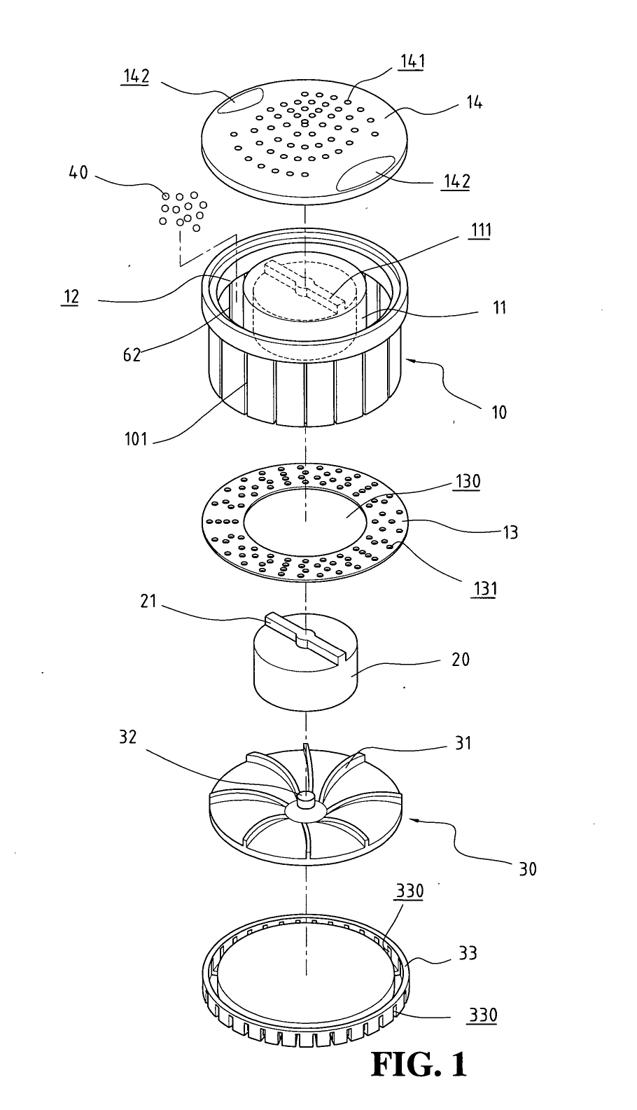

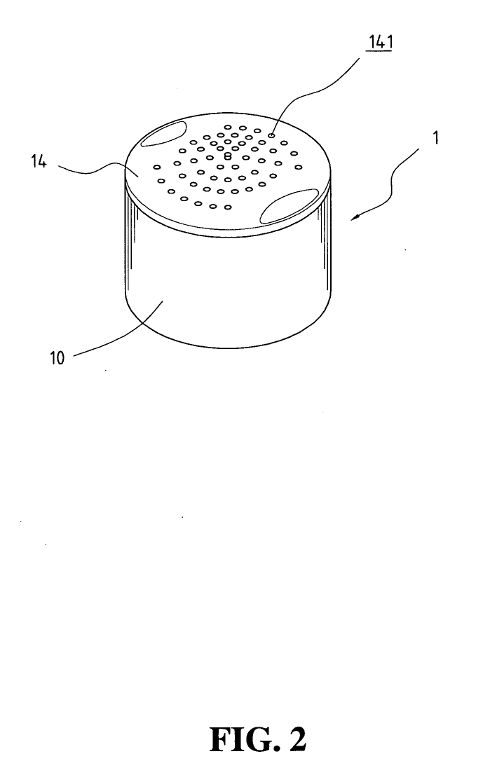

With reference to FIGS. 1 and 2, it is noted that the scent release massager in accordance with the present invention includes a container 10, a motor 20 and a fan 30.

The container 10 is hollow and includes multiple slits 101 defined in a side face of the container 10 to allow an interior of the container 10 to communicate with surrounding air, a bottom opening and a top opening respectively and oppositely defined therein, a hollow casing 11 received in the container 10 and provided with an opening defined in a bottom of the casing 11 to communicate with the bottom opening of the container 10 and a recess 111 defined in an inner face of a top face of the casing 11, an annular recess 12 defined between an outer periphery of the casing 11 and an inner periphery of the container 10, and a baffle 13 provided to a bottom of the bottom opening of the container 10 and having a through hole 130 centrally defined therethrough and multiple apertures 131 through the baffle 13 around the through hole 130.

The motor 20 has a elongated protrusion 21 formed on a top face of the motor 20 to correspond to the recess 111 of the casing 11. The fan 30 is securely engaged with the bottom of the container 10 via a securing ring 33 to close the bottom opening of the container 10 and has multiple blades 31 divergently extending outward therefrom and an axle 32 formed on a joint of the blades 31. The securing ring 33 has multiple air slits 330 defined in a periphery of the securing ring 33 such that air is able to exchange between the interior and exterior of the container 10. The securing ring 3 is threadingly connected to the bottom of the container 10.

With reference to FIG. 2, it is noted that after the scent release massager of the present invention is assembled, the elongated protrusion 21 of the motor 20 is received in the corresponding recess 111 of the casing 11 and the baffle 13 is mounted on the bottom peripheral edge defining the bottom opening of the container 10. Preferably the baffle 13 is a screen. Then the fan 30 is securely engaged with the peripheral edge defining the bottom opening of the container 10 to close the bottom opening of the container 10 via the securing ring 33, wherein the axle 32 is operably connected to the motor 20. Finally, a cover 14 having multiple second apertures 141 defined through the cover 14 and two recessed areas 142 respectively defined in two opposite points on a top face of the cover 14 is applied to close the top opening of the container 10. The two recessed areas 142 are able to facilitate holding of the cover 14. Therefore, essence particles 40 are able to be placed on top of the baffle 13 so as to be supported by the baffle 13 inside the container 10 before the cover 14 is applied to close the top opening of the container 10.

When the scent massager is in operation, it is noted that after the motor 20 is activated inside the casing 11, the blades 31 will be rotated to drive air into the interior of the container 10 via the air slits 330 defined in the securing ring 33. Vibration caused by the activation of the motor 20 will also drive the baffle 13 to vibrate as well. In the meantime, because ambient air is sucked into the interior of the container 10 via the air slits 330, the air inside the container 10 will be forced to escape from the slits 101 and the second apertures 141. While the interior air in the container 10 is flowing out of the container 10, the scent from the scent particles 40 is also carried by the out-flowing air. Thus, people surrounding the scent release massager of the present invention is able to smell the spiritually soothing smell. In addition, the user is able to apply the massager of the present invention onto a particular area of the body to proceed muscle relaxation process. As a result, both the spiritual and physical relaxation are achieved by a single device.

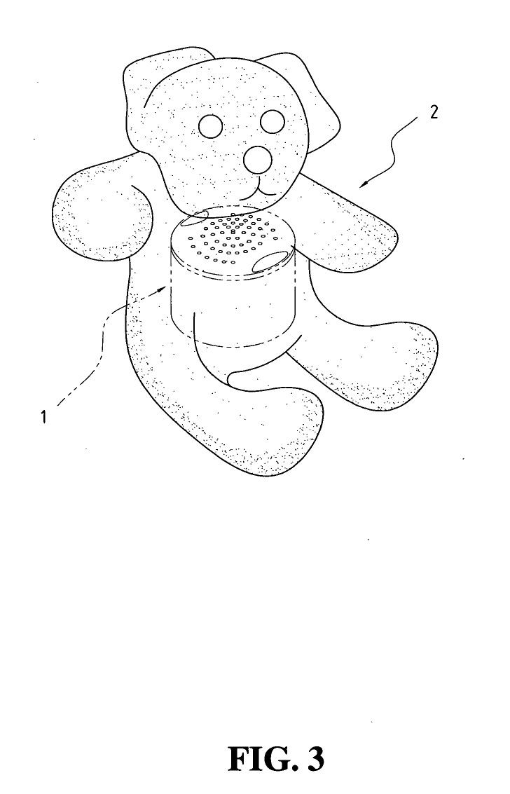

With reference to FIG. 3, it is noted that the user is able to place the scent release massager 1 of the present invention into a stuffed toy 2 and hold the stuffed toy 2. Thus when the scent release massager is activated, the user is able to have both the spiritual and physical relaxation. Besides, when the scent release massager is not in use, the scent release massager with the stuffed toy 2 is able to function as a decorator to increase the environment atmosphere.

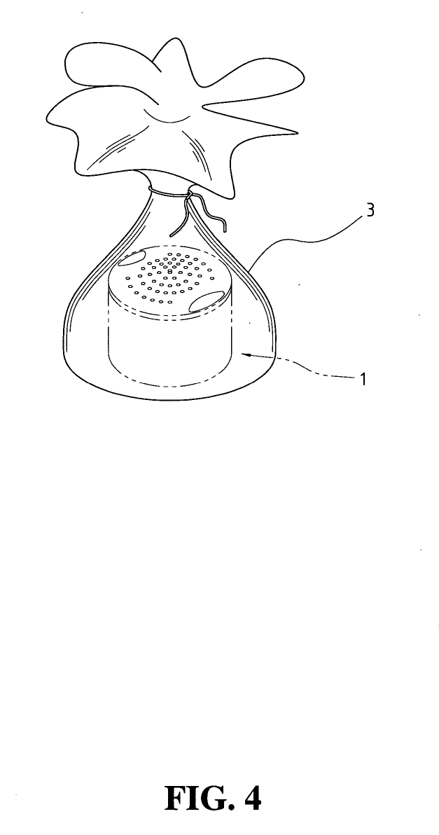

With reference to FIG. 4, it is noted that the user is able to place the scent release massager 1 of the present invention into a wrapping textile 3 and hold the wrapping textile 3. Thus when the scent release massager is activated, the user is able to have both the spiritual and physical relaxation. Besides, when the scent release massager is not in use, the scent release massager with the wrapping textile 3 is able to function as a decorator to increase the environment atmosphere.

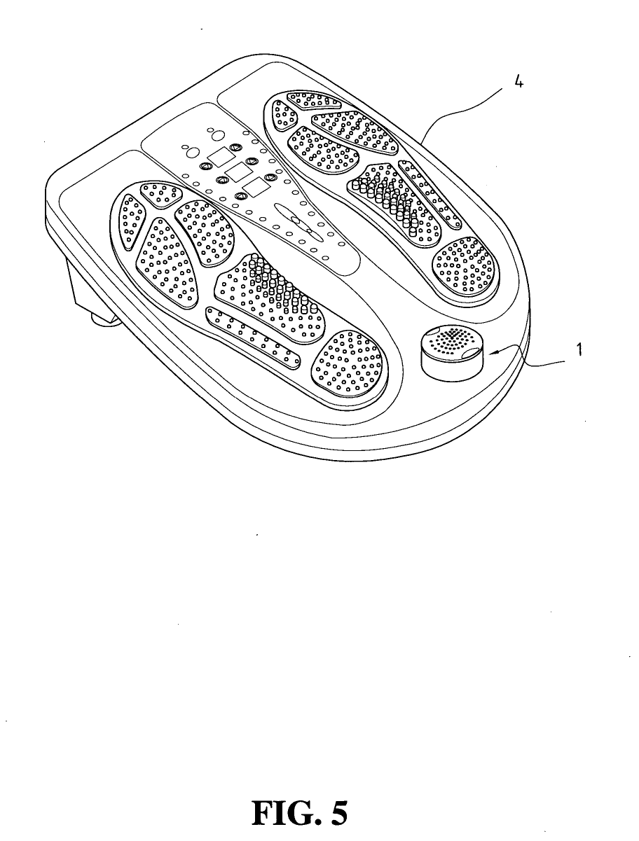

With reference to FIG. 5, it is noted that the user is able to place the scent release massager 1 of the present invention into a foot massager 4. Thus when the user is having a feet massaging effect using the foot massager 4, the scent release massager is also activated, the user is able to have both the spiritual and physical relaxation.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A scent release massager comprising:
a container with multiple slits defined in a side wall thereof, a bottom opening and a top opening;
a casing received in the container and supported by a fan which is securely engaged with a bottom periphery defining the bottom opening and has blades divergently extending outward and an axle formed on a joint of the blades;
a motor securely received in the casing to be supported by the fan, wherein the fan is operably connected to the motor;
a baffle sandwiched between the fan and the bottom periphery defining the bottom opening of the container and having multiple apertures defined through the baffle so as to support thereon scent particles; and
a cover detachably connected to the container to close the top opening of the container,
whereby when the motor is activated, the blades of the fan is driven to rotate so that scent from the scent particles is able to escape from the slits of the container.

2. The scent release massager as claimed in claim 1, wherein the casing has a recess defined in an inner face of a top face of the casing to correspond to and receive therein an elongated protrusion which is formed on top of the motor so that the motor is securely received in the casing.

3. The scent release massager as claimed in claim 2, wherein the fan has an axle connected to the motor so that the fan is able to be driven by the motor.

4. The scent release massager as claimed in claim 1, wherein a securing ring is applied to secure engagement between the fan and the bottom periphery defining the bottom opening of the container.

5. The scent release massager as claimed in claim 2, wherein a securing ring is applied to secure engagement between the fan and the bottom periphery defining the bottom opening of the container.

6. The scent release massager as claimed in claim 3, wherein a securing ring is applied to secure engagement between the fan and the bottom periphery defining the bottom opening of the container.

7. The scent release massager as claimed in claim 6, wherein an annular recess is defined between an inner periphery of the casing and an outer periphery of the casing to allow air exchange between an interior and an exterior of the container via the slits.

8. The scent release massager as claimed in claim 1, wherein the cover has multiple second apertures and two recessed areas defined in two opposite points of the cover to facilitate holding of the cover.

9. The scent release massager as claimed in claim 2, wherein the cover has multiple second apertures and two recessed areas defined in two opposite points of the cover to facilitate holding of the cover.

10. The scent release massager as claimed in claim 3, wherein the cover has multiple second apertures and two recessed areas defined in two opposite points of the cover to facilitate holding of the cover.

11. The scent release massager as claimed in claim 4, wherein the cover has multiple second apertures and two recessed areas defined in two opposite points of the cover to facilitate holding of the cover.

12. The scent release massager as claimed in claim 5, wherein the cover has multiple second apertures and two recessed areas defined in two opposite points of the cover to facilitate holding of the cover.

13. The scent release massager as claimed in claim 6, wherein the cover has multiple second apertures and two recessed areas defined in two opposite points of the cover to facilitate holding of the cover.

14. The scent release massager as claimed in claim 7, wherein the cover has multiple second apertures and two recessed areas defined in two opposite points of the cover to facilitate holding of the cover.

15. The scent release massager as claimed in claim 14 further comprising a stuffed toy to receive therein the scent massager.

16. The scent release massager as claimed in claim 14 further comprising a foot massager to receive therein the scent release massager.

17. The scent release massager as claimed in claim 16, wherein the baffle is a screen.
